(19) United States
(12) Patent Application Publication
(10) Pub. No.: US 2024/0382235 A1
(43) Pub. Date: Nov. 21, 2024

(54) Devices To Be Actuated Within The Uterus To Provide Suction For Treating Uterine Bleeding

(71) Applicant: Stryker Corporation, Kalamazoo, MI (US)

(72) Inventors: Brian MacLachlan, Norton Shores, MI (US); Robert L. Esser, Rockford, MI (US); Laura Frank, Greer, SC (US); Patrick Lafleche, Kalamazoo, MI (US); Leif Norland, St. Joseph, MI (US); Lucas Wade, Austin, TX (US); Lauren R. West, Cedar Park, TX (US); John P. Bernero, Round Rock, TX (US); Christy Sepulveda, Austin, TX (US); Spencer Gage VanSickle, Plainwell, MI (US)

(73) Assignee: Stryker Corporation, Kalamazoo, MI (US)

(21) Appl. No.: 18/687,029

(22) PCT Filed: Aug. 26, 2022
(86) PCT No.: PCT/US2022/041636

§ 371 Date: Feb. 27, 2024

Related U.S. Application Data
(60) Provisional application No. 63/237,611, filed on Aug. 27, 2021.

(51) Int. Cl. A61B 17/42; A61B 17/00; A61M 1/00

(52) U.S. Cl. CPC A61B 17/4241; A61M 1/682; A61M 1/743; A61B 2017/00561; A61B 2017/4225; A61B 2217/005; A61M 2210/1433

(57) ABSTRACT

Devices for treating uterine bleeding. An actuator is configured to receive an input to deploy an intrauterine portion of the device to assume a larger outer profile within the uterus. The actuator may be coupled to a device body and configured to receive a pull input, a push input, or a twist input. The intrauterine portion may include flexible opposing members, rotatable tines, a segmented hook, an adjustable loop, or compressible foam. The intrauterine portion defines a lumen and suction ports configured to draw suction within the uterus. Indicia may be provided on the actuator to indicate an extent by which the intrauterine portion has been deployed within the uterus. An orientation of the actuator may correspond to a direction of deployment of the intrauterine portion. The device may include valves and an auxiliary suction source configured to provide suction with the device decoupled from a vacuum source.

### PRIORITY CLAIM

This application claims priority to and all the benefits of U.S. Provisional Patent Application No. 63/237,611, filed on Aug. 27, 2021, the entire contents of which are hereby incorporated by reference.

### BACKGROUND

Excessive bleeding during or following childbirth is a major cause of maternal morbidity. Postpartum hemorrhage is an especially severe condition in which weak or absent contractions following childbirth fail to put sufficient pressure on bleeding vessels, a phenomena known as uterine atony.

It is known to treat postpartum hemorrhage by deploying a device within the uterus. The use of these devices with the application of a vacuum can counteract uterine atony by removing excess blood and inducing contractions. Owing to the relative dimensions of the uterus and the body opening through which the device needs to be inserted, there is a need in the art for a vacuum-based device that may be more intuitively inserted into and deployed within the uterus to achieve maximum effect. Further, at certain times, it may be necessary to transport the patient with the device in situ, which can undesirably require attending medical personnel decouple the device from the vacuum source such that treatment is at least temporarily halted. It would be desirable to obviate or limit such instances. Additional shortcomings in the art that are overcome by the present invention are also disclosed.

### SUMMARY

The present disclosure is directed to devices, systems, and methods for treating uterine bleeding with application of a vacuum. An intrauterine device is coupled to an end of a suction line to be arranged in fluid communication with a vacuum source. The device includes an intrauterine portion configured to be positioned within the uterus. The intrauterine portion may have a shape or an outer profile in a deployed configuration that is at least partially complementary to anatomical dimensions of the uterus. The intrauterine portion may be positioned within the uterus through a body opening. The intrauterine device can be selectively actuated to alter the shape and/or size of the intrauterine portion.

The intrauterine device includes the intrauterine portion, and a device body to which the intrauterine portion is coupled. A vacuum connector is coupled to the device body and configured to be removably coupled with the suction line. The intrauterine portion defines at least one lumen. The intrauterine portion may include opposing members extending from a collar. The opposing members may be coupled to one another to form a loop in the deployed configuration. The opposing members may be resilient to return to an original or natural state in the absence of forces on the device. The loop formed by the opposing members define respective central sections between a distal end and the collar. The central sections may be biased to engage or be disposed adjacent to one another in an undeployed configuration. The loop may be initially “closed” by default. An outer profile of the intrauterine portion in the undeployed configuration may be narrower than the collar and/or narrower than the device body.

The intrauterine portion defines suction ports. The suction ports may be arranged on inner or outer surfaces of the opposing members. The suction ports are in fluid communication with the lumen defined by the opposing members. The device is configured to be moved from the undeployed configuration to the deployed configuration to expand the outer profile of the intrauterine portion to assume additional volume within the uterus. The central sections of the opposing members are configured to be spaced from one another in the deployed configuration for the suction to be drawn through the suction ports. An actuator is coupled to the intrauterine portion, and to the device body. The actuator may be a handle slidably disposed within a hub extending from the device body. The hub may extend radially from the device body at a radial orientation corresponding to a direction of deployment of the intrauterine portion.

The actuator is configured to receive an input from the user to move the intrauterine portion between the undeployed configuration and the deployed configuration. The actuator may be a manual actuator in which the handle is shaped to receive a pull input to draw proximally the distal end of the intrauterine portion and flex outwardly the central sections and increase the outer profile of the intrauterine portion. The actuator may be movable between first and second positions corresponding to the undeployed and deployed configurations, respectively, or any positions therebetween. The actuator may include a lock feature, and indicia. The lock feature is configured to permit the actuator to be selectively locked and released in one of several positions between and including the first and second positions. The indicia may provide the user with information as the extent of deployment. Alternatively, the actuator may be a push actuator or a twist actuator. In another variant, an electronic actuator may be used in which an electronic switch may be actuated to operate a motor to deploy the intrauterine portion.

A baffle may be coupled to the intrauterine portion. The baffle may be positioned between the opposing members, and extend between the distal end and the collar. The baffle engages the opposing members in the undeployed configuration so as to occlude the suction ports. The baffle is positioned between and spaced apart from the central sections of the opposing members in the deployed configuration, thereby the suction ports being patent to draw the suction on the uterus.

The device includes the cervical seal. The cervical seal may be a bladder configured to be inflated to expand. Alternatively, the cervical seal may include a casing, and a resiliently compressible body within the casing that is configured to be squeezed for positioning within the cervical os, and thereafter return to a natural state to form the seal. In another variant, opposing axial ends of the cervical seal may be urged towards one another to cause the cervical seal to flare outwardly to form the seal in a manner akin to the intrauterine portion. The actuator may be operably coupled to the cervical seal so as to simultaneously deploy the cervical seal and the intrauterine portion.

The device may include an auxiliary suction source coupled to the device body and in fluid communication with the lumen. In one example, the auxiliary suction source is a Jackson-Pratt bulb or drain. First and second valves may be one-way valves with the first valve position distal to the auxiliary suction source and the second valve disposed proximal to the auxiliary suction source. The vacuum connector may be disposed proximal to the second valve. The resilience of the Jackson-Pratt bulb causes the suction to be drawn after release of a manual input, and further provides a reservoir in which the fluids may be collected. A relief port may be coupled to the device body and positioned distal to the first valve.

In certain implementations, a twist actuator may be coupled to the device body. The intrauterine portion may include at least two tines. The tines may be formed as loops with each of the tines defining a lumen, and suction ports in fluid communication with the lumen. The actuator may include one or more collars with each of the collars coupled to a respective one of the tines. The input to the actuator imparts a corresponding rotation of one or more of the collars, and therefore one or more of the tines. In the undeployed configuration, the tines may be arranged to lie in a single reference plane in the undeployed configuration such that the tines assume a “flattened” profile. The tines may be splayed outside of the single reference plane in the deployed configuration.

In certain implementations, the actuator may be a slider movably coupled to the device body. The actuator may eject the intrauterine portion from within a bore of the device. The intrauterine portion may include a plurality of segments formed by a plurality of notches that further define a spine. The spine may define the lumen, and further define the suction ports in fluid communication with the lumen. Alternatively, the segments may be discrete components pivotably joined to one another. The intrauterine portion may be a biased or pre-stressed member configured to at least partially bend and/or curl when ejected from the bore. With the slider in the first position, the segments are constrained by the bore in a straight configuration. The slider is moved to the second position to expose the intrauterine portion beyond the distal end, after which the segments pivot relative to one another into a hook or other suitable shape.

In certain implementations, the intrauterine portion is an adjustable loop. The loop defines the lumen, and the suction ports in fluid communication with the lumen. The loop may include a first opposing member fixedly coupled to the device body, and a second opposing member that is slidably within the device body. The slider is configured to be translated relative to the device body to cause the loop to be further exposed beyond the distal end. In one variant, both the first and second opposing members are slidably within the device body with the actuator coupled to the first and second opposing members. Alternatively, more than slider may be coupled to a respective one of the first and second opposing members.

In certain implementations, the device includes an applicator defining a bore sized to receive a head of the intrauterine portion. The head may be formed from a porous medium, such as a resiliently compressible biocompatible foam. The head may be coupled to an inner tube defining a lumen in fluid communication with the foam. The head is compressible so as to be fully received within the applicator in the undeployed configuration. The actuator may be operably coupled to the inner tube. The actuator is configured to receive the input from the user to move from the first position to the second position to correspondingly move the inner tube and expose the head distally beyond the applicator. The resilience of the foam causes the outer profile of the head to increase, thereby moving the intrauterine portion to the deployed configuration. The foam has absorbency to draw the blood into the head. Further, suction is drawn through the head, and additional suction may be drawn through the applicator.

In certain implementations, the opposing members may each terminate at a respective distal end. The opposing members may define channels with the suction ports disposed within the channels. The opposing members may each include a proximal end coupled to the collar, and a living hinge between the proximal end and the distal end to permit a distal section of the opposing members to pivot relative to a proximal section. In a first variant, the opposing members are formed or biased to be fully extended. The actuator is actuated to move the device to the undeployed configuration in which the distal sections pivot inwardly about the living hinges to create a narrower outer profile of the intrauterine portion. The input to the actuator may be removed, and the resiliency of the opposing members causes the distal sections to pivot outwardly about the living hinges for the opposing members to return to their natural state, thereby moving the device to the deployed configuration. In a second variant, the opposing members are biased for the distal sections to be folded inwardly. The actuator is actuated to cause the distal sections to pivot outwardly about the living hinges, thereby moving the device to the deployed configuration. The first and second opposing members may independently deployable.

In certain implementations, the actuator is actuated to selectively permit or prevent suction through one or more the suction ports to reduce clogging of the suction ports. The intrauterine portion may include a head defining the suction ports, and an inner tube rotatably disposed within the head. The inner tube may define a lumen configured to be arranged in fluid communication with the suction path, and one or more openings in fluid communication with the lumen. The openings are configured to be arranged in selective fluid communication with less than all of the suction ports. The discontinuation of the suction through the clogged suction port(s) may result in the blood clot becoming dislodged.

In certain implementations, the intrauterine portion includes a shell that defines a volume, and apertures in fluid communication with the volume. A head includes a face that defines the suction ports. The shell is coupled to the head to define the volume with the suction ports opening into the volume. The suction ports may be further defined by funnels extending inwardly from the face, and protrusions extending outwardly from the face. The shell is configured to provide a first barrier to capture larger blood clots. The funnels and/or the protrusions are configured to provide a second barrier to capture smaller blood clots.

### BRIEF DESCRIPTION OF THE DRAWINGS

FIG. 1 is a perspective view of a medical waste collection system with a manifold configured to be removably inserted into the medical waste collection system, and suction lines configured to be removably coupled to the two manifold. An intrauterine device is configured to be removably coupled to the suction line.

FIG. 2A is a perspective view of an implementation of the device with an intrauterine portion in an undeployed configuration.

FIG. 2B is a perspective view of the device of FIG. 2A with the intrauterine portion in a deployed configuration.

FIG. 3A is a perspective view of another implementation of the device with the intrauterine portion in the undeployed configuration.

FIG. 3B is a perspective view of the device of FIG. 3A with the intrauterine portion in the deployed configuration.

FIG. 4A is an elevation view of another implementation of the device with the intrauterine portion in the undeployed configuration.

FIG. 4B is an elevation view of the device of FIG. 4A with the intrauterine portion in the deployed configuration.

FIG. 5A is an elevation view of another implementation of the device with the intrauterine portion in the undeployed configuration.

FIG. 5B is an elevation view of the device of FIG. 5A with the intrauterine portion in the deployed configuration.

FIG. 6A is an elevation view of another device with an intrauterine portion in a partially deployed configuration.

FIG. 6B is an elevation view of the device of FIG. 7A with the intrauterine portion in the deployed configuration.

FIG. 7 is a perspective view of another implantation of device with the intrauterine portion in the deployed configuration.

FIG. 8A is a perspective view of the intrauterine portion of another implementation of the device.

FIG. 8B are elevation views of the intrauterine portion of FIG. 8A.

FIG. 8C is sectional view of the intrauterine portion of FIGS. 8A-8B with an inner tube in a first rotational orientation relative to a head.

FIG. 8D is sectional view of the intrauterine portion of FIGS. 8A-8B with the inner tube in a second rotational orientation relative to the head.

FIG. 9 is a perspective view of the intrauterine portion of another implementation of the device.

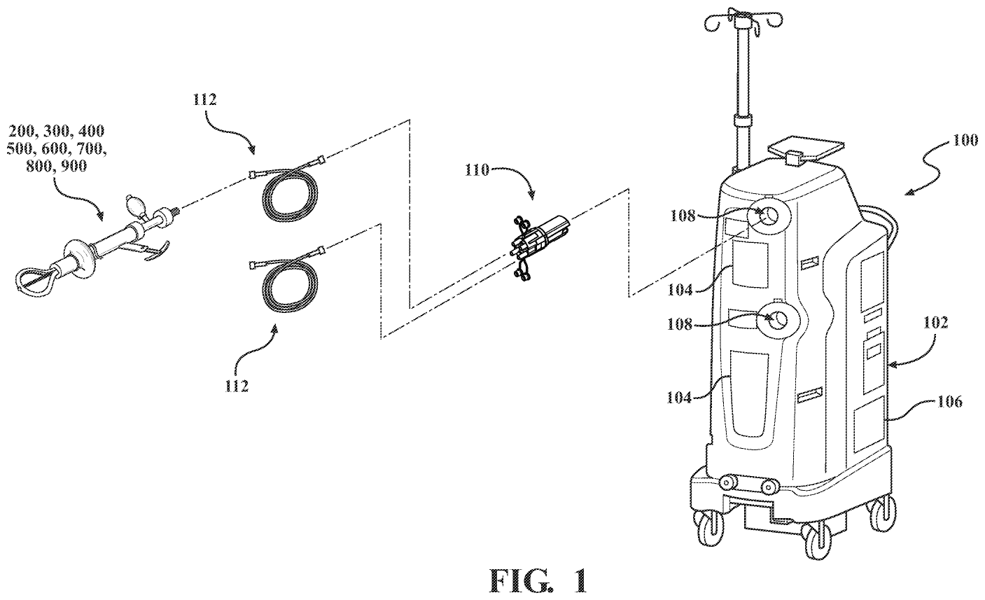

### DETAILED DESCRIPTION

The present disclosure is directed to devices, systems, and methods for treating uterine bleeding with a vacuum source. The vacuum source may be realized through any suitable means, such as a hospital-integrated vacuum system. Another exemplary arrangement is a medical waste collection system 100 sold under the tradename Neptune by Stryker Corporation (Kalamazoo, Mich.) in which a waste container 104 and a vacuum source 106 is supported on a chassis 102. The medical waste collection system 100 collects and stores the medical waste to be operably coupled with a docking station through which the medical waste is emptied. At least one receiver 108 may be supported on the chassis 102 and sized to removably receive a manifold 110 to which at least one suction line 112 is removably coupled. Suitable construction and operation of several subsystems of the medical waste collection system 100 are disclosed in commonly-owned United States Patent Publication No. 2005/0171495, published Aug. 4, 2005, International Publication No. WO 2007/070570, published Jun. 21, 2007, International Publication No. WO 2014/066337, published May 1, 2014, and commonly-owned International Publication No. WO 2017/112684, published Jun. 29, 2017, the entire contents of each being hereby incorporated by reference.

An intrauterine device 200, 300, 400, 500, 600, 700, 800, 900 is coupled to an end of the suction line 120 opposite the manifold 124 to be arranged in fluid communication with the vacuum source 106. Implementations of the device include an intrauterine portion configured to be positioned within the uterus, after which blood and other bodily fluids are drawn from within the uterus by suction provided by the vacuum source 106. Further, the intrauterine portion may have a shape or an outer profile in a deployed configuration that is at least partially complementary to anatomical dimensions of the uterus to maximize the effect of suction and induce contractions. The intrauterine portion may be positioned within the uterus through a body opening, namely the cervix or a caesarian opening. As mentioned, it is difficult to employ a device that is sufficiently narrow for insertion through the body opening but sufficiently large to assume an appreciable volume within the uterus. The present disclosure addresses the aforementioned challenge by employing implementations of the intrauterine device that can be selectively actuated to alter the shape and/or size of the intrauterine portion. For example, the shape and size of the intrauterine portion may be deployed to engage or conform to the fundus of the uterus to promote hemostasis. Further advantages of the various implementations of the intrauterine device will also be realized.

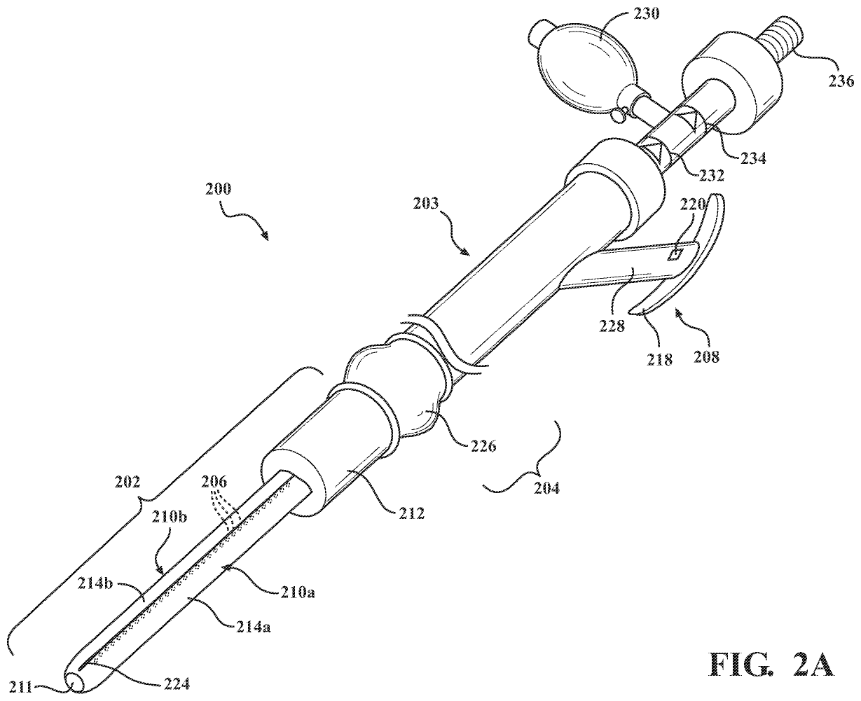

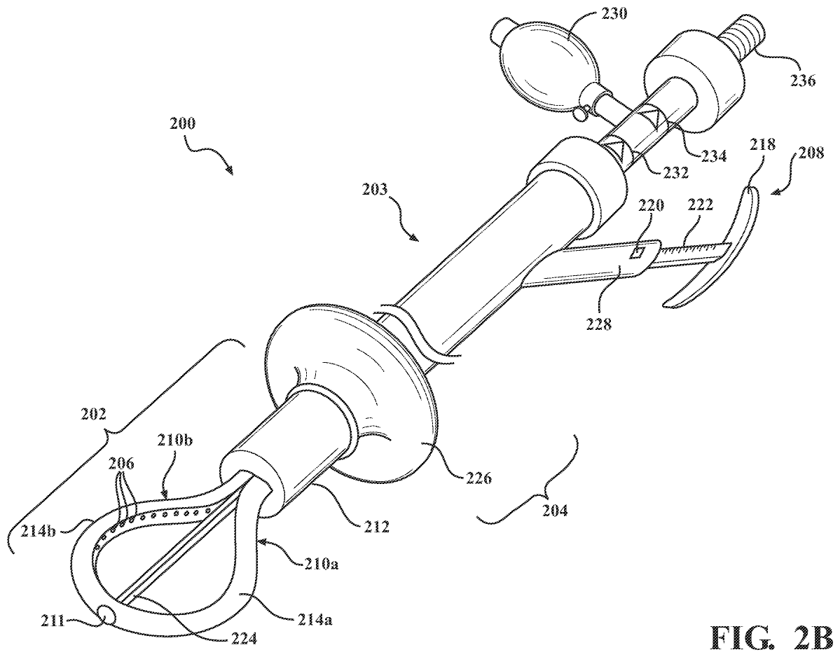

Referring to FIGS. 2A and 2B, the intrauterine device 200 includes the intrauterine portion 202, and a device body 203 to which the intrauterine portion 202 is coupled. The device body 203 provides a housing defining an interior through which tubing and other subcomponents the device 200 may be disposed. A vacuum connector 236 may be coupled to the device body 203 and configured to be removably coupled with the suction line 120 to establish fluid communication between the device 200 and the vacuum source 106. The vacuum connector 236 may be rigidly secured to the device body 203 to define a proximal end of the device 200, or an intermediate tube (not shown) may extend from the device body 203 and include the vacuum connector 236. Other locations of the vacuum connector 236 are contemplated so as to facilitate line management. The device body 203 may be elongate and function as a handle for manipulating and deploying the device 200 in a manner to be described. For example, the device body 203 is may be cylindrical in shape and have a length sufficient for certain features of the device 200 to be positioned external to the vagina (and the patient) so as to be operable by the user. The device body 203 may include contours, geometries, and/or texturing disposed along portions thereof to facilitate handling and manipulation of the device 200.

The intrauterine portion 202 may define at least one lumen (not identified). More particularly, the intrauterine portion 202 may include opposing members 210a, 210bextending from a collar 212 with one or both the opposing members 210a, 210b defining the lumen. The opposing members 210a, 210b may be coupled to one another at or near respective distal ends to define a distal end 211 of the device 200. In such an arrangement, the opposing members 210a, 210b form a loop in the deployed configuration. FIGS. 2A and 2B show two opposing members 210a, 210b, however, more than two opposing members 210a, 210b are contemplated in which the opposing members 210a, 210b may be deployed radially in a triangular (three), cruciform (four), stellate (five), or other arrangement.

The opposing members 210a, 210b may be formed from resilient biocompatible material. The material may be sufficiently flexible to permit flexion in instances where the distal end 211 is engaging tissue, but sufficiently resilient to return to an original or natural state in the absence of forces on the device 200. The distal end 211 of the device 200 may include a blunt tip to avoid trauma to tissue during insertion and deployment of the device 200. The blunt tip may also be formed from a resilient biocompatible material. The distal end 211, or another portion of the intrauterine portion 202, may be radiopaque or include a radiopaque marker to be visualizable on fluoroscopy. In such instances, the position of the device 200 within the uterus may be visually confirmed and adjusted accordingly, if desired.

The intrauterine portion 202, and more particularly the opposing members 210a, 210b, have a length sufficient for the distal end 211 to be positioned adjacent the fundus of the uterus with a cervical seal 226 sealing the cervix. Further, the loop formed by the opposing members 210a, 210b define respective central sections 214a, 214b between the distal end 211 and the collar 212. The central sections 214a, 214b may be biased to engage or be disposed adjacent to one another in an undeployed configuration as shown in FIG. 2A. In other words, the central sections 240a, 214b are formed for the loop to initially be “closed” by default. As a result, the device 200 may be constructed such that, in the absence of forces on the device 200, the intrauterine portion 202 is sufficiently narrow for insertion through the body opening. An outer profile of the intrauterine portion 202 in the undeployed configuration may be narrower than the collar 212, and/or narrower than the device body 203.

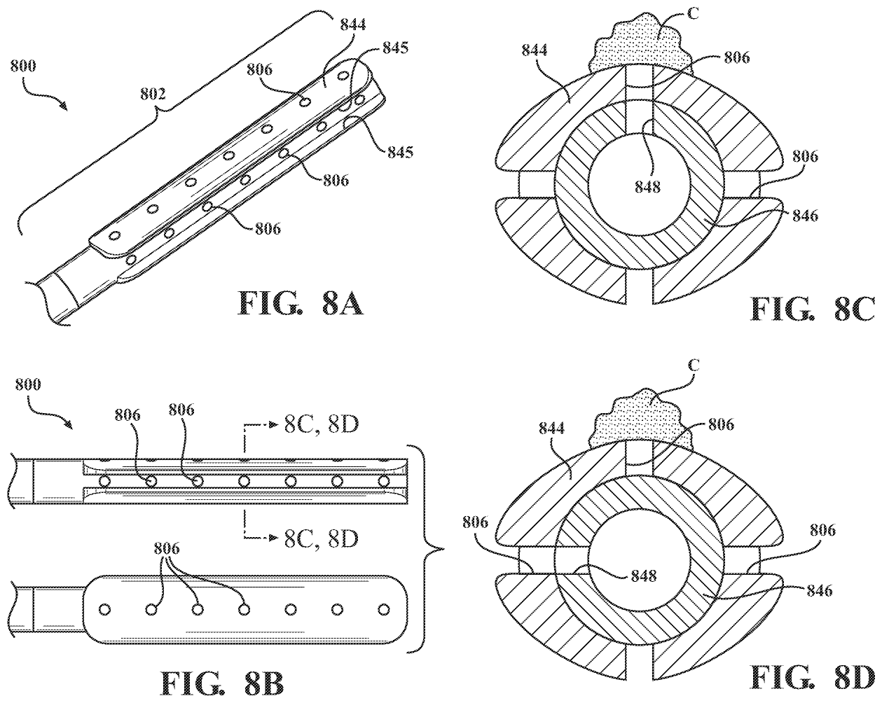

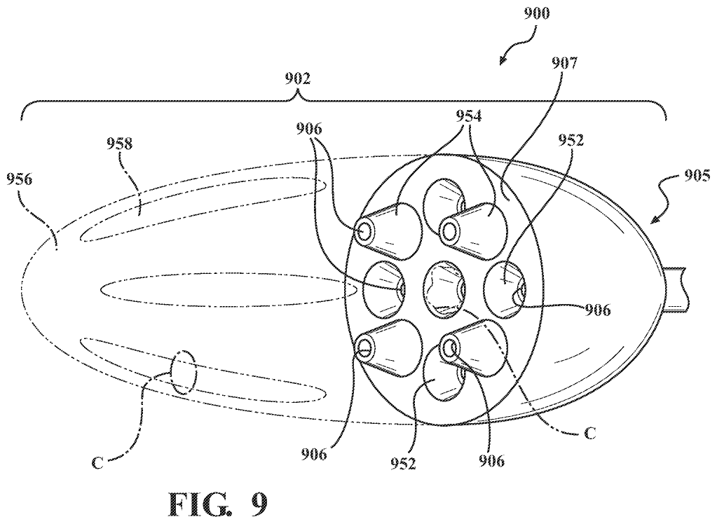

The intrauterine portion 202 defines suction ports 206. The suction ports 206 may be arranged on the inner surfaces of the opposing members 210a, 210b. The suction ports 206 may a linear array of circular holes, as shown, but other shapes and configurations may be provided. Other locations for the suction ports 206 are additionally or alternatively contemplated (see FIG. 8). The suction ports 206 are in fluid communication with the lumen defined by the opposing members 210a, 210b. As a result, the device 200 defines a suction path through the suction ports 206, the lumen, and the vacuum connector 236 to draw blood and other bodily fluids from the uterus to be collected in the medical waste collection system 100.

Owing to the narrow profile of the intrauterine portion 202 in the undeployed configuration, the intrauterine portion 202 may be inserted through the body opening in an intuitive manner. In other words, the user need not manipulate the intrauterine portion 202 (e.g., collapsing the loop) to be insertable through the body opening. Thereafter, the device 200 may be moved from the undeployed configuration to the deployed configuration to expand the outer profile of the intrauterine portion 202 to assume additional volume within the uterus, and preferably contact the fundus of the uterus to the extent possible. More particularly, the central sections 214a, 214b of the opposing members 210a, 210b are configured to be spaced from one another in the deployed configuration for the suction to be drawn through the suction ports 206, as reflected in FIG. 2B. To that end, an actuator 208 is coupled to the intrauterine portion 202, and to the device body 203. The actuator 208 may be a handle 218 slidably disposed within a hub 228 extending from the device body 203. The hub 228 may extend radially from the device body 203 at a radial orientation corresponding to a direction of deployment of the intrauterine portion 202. In other words, the user may visualize a radial position of the actuator 208—positioned external to the patient—and ascertain the direction in which the opposing members 210a, 210b are to deploy within the uterus. For example, FIG. 2B generally shows the actuator 208 extending laterally from a side of the device body 203, and the intrauterine portion 202 is correspondingly deployed laterally.

The actuator 208 is configured to receive an input from the user to move the intrauterine portion 202 between the undeployed configuration and the deployed configuration. FIGS. 2A and 2B show the actuator 208 being a manual actuator in which the handle 218 is shaped to receive a pull input to draw the handle 218 outwardly from within the hub 228. The handle 218 may be coupled to a pull wire (not shown) extending through the device body 203 and through the collar 212 to be operably coupled to the distal end 211 of the intrauterine portion 202. The movement of the handle 218 draws proximally the distal end 211 of the intrauterine portion 202. The opposing members 210a, 210b, and in particular the flexibility of the central portions 214a, 214b of the opposing members 210a, 210b, are flexed outwardly to open the loop. The outer profile of the intrauterine portion 202 is of greater size in the deployed configuration than the undeployed configuration. The outer profile of the intrauterine portion 202 in the deployed configuration may be greater in size than outer dimensions of the collar 212, and the device body 203. The opposing members 210a, 210b may be formed so that the central portions 214a, 214b flare outwardly to define a pear-shape or other suitable shape to the intrauterine portion 202, which may better complement the shape of the uterus.

The actuator 208 may be movable between first and second positions corresponding to the undeployed and deployed configurations, respectively, or any positions therebetween. In other words, the actuator 208 may be actuated to selectively deploy the intrauterine portion 202 to a desired extent. The desired extent of deployment may be based on the patient status, anatomical dimensions, or the like. For example, shortly after childbirth, the size of the uterus may be relatively pronounced, and therefore it may be desirable to fully deploy the intrauterine portion 202 to attempt to contact the fundus of the uterus and maximize the effect of suction. As the uterine atony is being successfully treated, the uterus may begin to contract, and therefore it is desirable to have the intrauterine portion 202 be deployed to a lesser extent. To that end, the actuator 208 may include a lock feature 220, and indicia 222. The lock feature 220 is configured to permit the actuator 208 to be selectively locked and released in one of several positions between the first and second positions. The lock feature 220 may be a switch, a detent, or other suitable mechanism for selectively maintaining the intrauterine portion 202 in the desired extent of deployment. The indicia 222 may provide the user with information as the extent of deployment. FIG. 2B shows the indicia 222 and graduated markings on the handle 218 that are configured to be exposed as the handle 218 is pulled relative to the hub 228. Numbers provided thereon may correspond to a width of the extent of deployment of the intrauterine portion 202.

As an alternative to a pull actuator, the actuator 208 may be a push actuator or a twist actuator. The twist actuator may include a wheel coupled to the device body 203, for example, concentrically disposed about the device body 203. Alternatively, the handle 218 may be rotatable within the hub 228. The pull wire may be operably coupled to the wheel or handle with a mandrel or other suitable mechanism. The twisting of the wheel or handle creates the tension on the pull wire to draw the distal end 211 of the intrauterine portion 202 proximally as described. It is alternatively contemplated that an electronic actuator may be used in which an electronic switch may be actuated. The switch may be in communication with a motor for tensioning the pull wire to deploy the intrauterine portion 202. Release of the switch may electronically maintain the intrauterine portion 202 in the desired extent of deployment. A digital readout may be disposed on the device body 203 to provide a numerical value of the extent of deployment of the intrauterine portion 202.

A baffle 224 may be coupled to the intrauterine portion 202 and configured to regulate the suction through the suction ports 206 with the intrauterine portion 202 in the undeployed configuration. As best shown in FIG. 2B, the baffle 224 may be positioned between the opposing members 210a, 210b, and more particularly extend between the distal end 211 and the collar 212. In the undeployed configuration, the baffle 224 engages the opposing members 210a, 210b so as to occlude the suction ports 206. The inner surfaces of the opposing members 210a, 210b engage opposing sides of the baffle 224 such that suction may not be drawn through the suction ports 206 in the undeployed configuration. Such a feature advantageously provides for the intrauterine portion 202 being insertable and removable through the body opening with the suction on—with lessened risk for tissue suction—as opposed to requiring the user turn off the vacuum source 106 to do so. Thereafter, the intrauterine portion 202 is moved from the undeployed configuration to the deployed configuration such that the baffle 224 is positioned between and spaced apart from the central sections 214a, 214b of the opposing members 210a, 210b, wherein the suction ports 206 become patent to draw the suction on the uterus. It is further contemplated that the pull wire of the actuator 208 may be coupled to a proximal end of the baffle 224, in which case the baffle 224 translates within the collar 212 to outwardly flex the central sections 214a, 214b of the opposing members 210a, 210b. It is understood that the baffle 224 is optional.

In order to ensure the maximum effect of suction through the suction ports 206 of the intrauterine portion 202 in the deployed configuration, the device 200 includes the cervical seal 226. The cervical seal 226 is part of a cervical portion 204 that is coupled to the intrauterine portion 202. The cervical portion 204 may also be coupled to the device body 203 and positioned between the device body 203 and the intrauterine portion 202. The cervical portion 204 is configured to be deployed for the cervical seal 226 to seal the cervical os with the intrauterine portion 202 positioned within the uterus. The cervical seal 226 may be a bladder configured to be inflated to expand. Alternatively, the cervical seal 226 may include a casing, and a resiliently compressible body within the casing that is configured to be squeezed for positioning within the cervical os, and thereafter return to a natural state to form the seal. In another example, opposing axial ends of the cervical seal 226 may be urged towards one another to cause the cervical seal 226 to flare outwardly to form the seal in a manner akin to the intrauterine portion 202. In such an example, the cervical portion 204 may include additional subcomponents (e.g., a slidable collar adjacent the proximal end of the cervical seal 226) configured to deploy the cervical seal 226. It is contemplated that the actuator 208 may be operably coupled to the cervical seal 226 so as to simultaneously deploy the cervical seal 226 and the intrauterine portion 202.

Existing systems may require the attending medical personnel decouple the device from the vacuum source during patient transport within a medical facility. Such instances undesirably suspend treatment. The device 200 of the present disclosure overcomes such a shortcoming by providing an auxiliary suction source 230 configured to establish or maintain at least some suction within the uterus during patient transport. In other words, the auxiliary suction source 230 may receive another input from the user to maintain the suction through the intrauterine portion 202 when the suction line 112 is decoupled from the vacuum connector 236. The auxiliary suction source 230 may be coupled to the device body 203 and in fluid communication with the lumen. In one example, the auxiliary suction source 230 is a Jackson-Pratt bulb or drain. The Jackson-Pratt bulb is a compact, lightweight, and low-cost subcomponent whose operation may be familiar to users. First and second valves 232, 234 may be one-way valves with the first valve 232 position distal to the auxiliary suction source 230 and the second valve 234 disposed proximal to the auxiliary suction source 230. The vacuum connector 236 may be disposed proximal to the second valve 234. Therefore, in instances where the suction tube is connected to the vacuum connector 236, the suction drawn by the vacuum source 106 opens the first and second valves 232, 234. In instances where the suction tube is disconnected from the vacuum connector 236, and the input is provided to the auxiliary suction source 230 (e.g., the Jackson-Pratt bulb is squeezed), the suction drawn by the auxiliary suction source 230 opens the first valve 232 but the second valve 234 remains closed. The blood and other bodily fluids are drawn through the intrauterine portion 202, through the first valve 232, and into the auxiliary suction source 230. More particularly, the resilience of the Jackson-Pratt bulb causes the suction to be drawn, and further provides a reservoir in which the fluids may be collected. Once the Jackson-Pratt bulb has resiliently returned to its natural state, and perhaps at least partially filled with collected fluids, the user may provide still another input to the Jackson-Pratt bulb. Owing to the one-way nature of the first and second valves 232, 234 and their respective positions relative to the auxiliary suction source 230, the first valve 232 is closed and second valve 234 is opened by the positive pressure from squeezing the Jackson-Pratt bulb. The fluids are urged proximally through the second valve 234 and the vacuum connector 236 be collected in a disposable bag that may be removably coupled to the vacuum connector 236 during transport of the patient. Not only are fluids emptied from the Jackson-Pratt bulb, but also the input simultaneously primes the auxiliary suction source 230 with additional suction. Once the patient has been transported to the desired location, one that is near to a medical waste collection system 100, the disposable collection bag may be decoupled from the vacuum connector 236 and another suction tube coupled to the device 200. The vacuum source 106 may be operated or reinitiated with little disruption in treatment. A relief port (not identified) may be coupled to the device body 203 and positioned distal to the first valve 232. The relief port is configured to provide for loss of suction if the suction level exceeds a predetermined threshold.

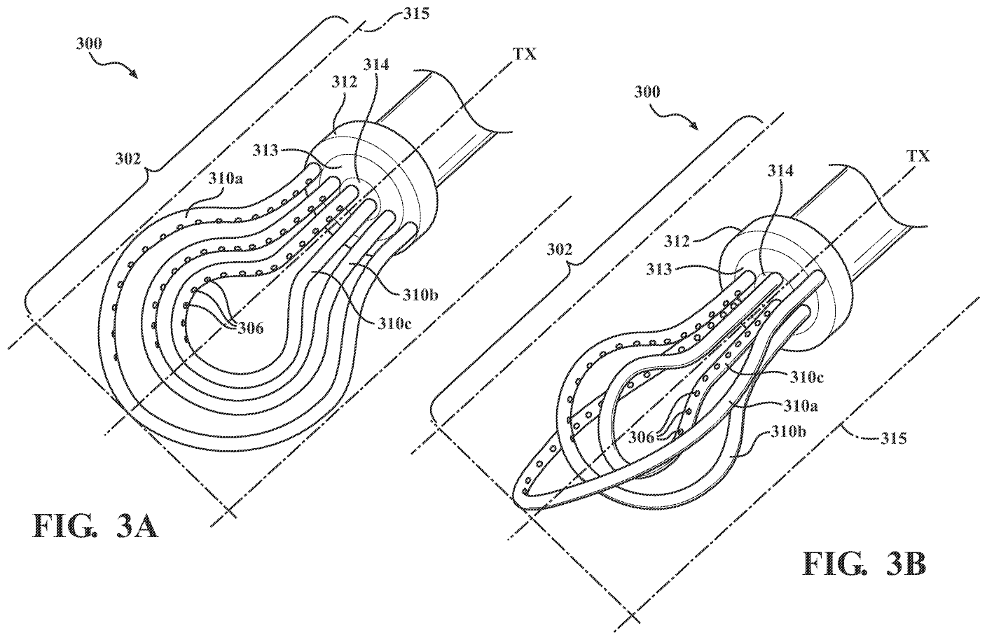

Referring now to FIGS. 3A and 3B, another implementation of the device 300 is shown in which intrauterine portion 302 is configured to be actuated via rotation. A twist actuator may be coupled to the device body (not shown), or alternatively another type of actuator may be mechanically or electromechanically converted into a rotational actuation of the intrauterine portion 302. The intrauterine portion 302 may include at least two tines 310. The illustrated implementation shows three tines 310a, 310b, 310c, but more or less are contemplated. An outer one of the tines 310a is larger than a middle one of the tines 310b which is larger than an inner one of the tines 310c. The tines 310 are formed as loops each with a generally straight proximal section that flares outwardly into a generally semicircular distal section. At least a distal end of the outer one of the tines 310a is rounded to provide a blunt end to avoid trauma to tissue during insertion and deployment of the device 300. Other shapes are contemplated so as to be complementary to the shape of the uterus. Likewise, the tines 310 may be similarly contoured, as shown, or each of the tines 310 may have a different contour or geometry. The tines 310 may be formed from resilient biocompatible material to permit a limited amount of flexion in instances where the device 300 is engaging tissue.

Each of the tines 310 defines a lumen, and suction ports 306 in fluid communication with the lumen. The lumens of the tines 310 may be merged within the device 300, for example within the device body, to form a single suction path. Alternatively, each of the lumens may remain fluidly separate, and valves may be provided within the device 300 that are configured to independently control suction through each of the tines 310. For example, it may be desirable to provide for a lower level of suction through the outer one of the tines 310a, as it is positioned closer to tissue within the uterus. By contrast, the inner one of the tines 310c may be operated at a higher level of suction where additional amounts of blood and bodily fluids may accumulate within the uterus. Additionally or alternatively, the suction through individual tines 310 may be selectively activated or deactivated based on patient status and other clinical considerations.

The actuator may include one or more collars 312, 313, 314. Each of the collars 312, 313, 314 are coupled to a respective one of the tines 310a, 310b, 310c. The collars 312, 313, 314 are rotatable relative to one another. To facilitate the relative rotation, the device body may include one or more coaxially arranged housings each coupled to a respective wheel. Rotation of a select one or more of the wheels imparts a corresponding rotation of one or more of the collars 312, 313, 314, and therefore one or more of the tines 310a, 310b, 310c. The tines 310 may be unidirectionally rotatable with a ratchet-like mechanism preventing rotation in an opposite direction, or the tines 310 may be bidirectionally rotatable. More particularly, the collars 312, 313, 314 are rotatable about an axis TX from a first orientation in which the intrauterine portion 302 is in the undeployed configuration (see FIG. 3A) to a second orientation in which the intrauterine portion 302 is in the deployed configuration (see FIG. 3B). In the undeployed configuration, the tines 310 may be arranged to lie in a single reference plane 315 such that the tines 310 are arranged in a flattened configuration. In the flattened configuration, the intrauterine portion 302 may more easily be inserted into and manipulated inside the uterus. In the deployed configuration, the at least two tines 310 are arranged to be splayed outside of the single reference plane 315. With the tines 310 splayed outside of the single reference plane 315 facilitates the intrauterine portion 302 to assuming a volume of and/or contacting the uterus in three dimensions. The wheels or another actuator may include respective locking mechanisms to maintain the angular orientation of the corresponding one of the tines 310. The wheels or other actuators may also include indicia corresponding to the angular orientation of the tines 310 within the anatomy. It is understood that the device 300 may include the cervical portion (e.g., cervical portion 204, 404, 504, 604), and additional features of other implementations of the device (e.g., auxiliary suction source 230, etc.).

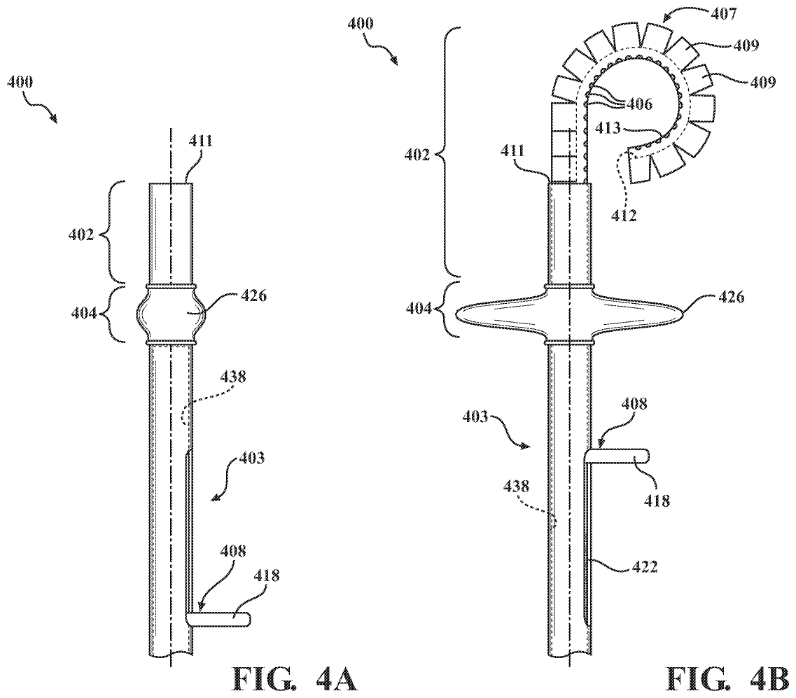

Referring now to FIGS. 4A and 4B, another implementation of the device 400 is shown in which the intrauterine portion 402 is configured to be actuated via a push input. The actuator 408 may be a slider 418 movably coupled to the device body 403. The actuator 408 is configured to receive an input to eject the intrauterine portion 402 from within a bore 438 of the device 400 in a manner to be described. The slider 418 may be moveable along a longitudinal axis of the device 400 from a first position in which the intrauterine portion 402 is in the undeployed configuration (see FIG. 4A) to a second position in which the intrauterine portion 402 is in the deployed configuration (see FIG. 4B).

The intrauterine portion 402 may include a plurality of segments 409. The segments 409 may be formed by a plurality of notches that further define a spine 413. Alternatively, the segments 409 may be discrete components pivotably joined to one another through hinge-like joints. The spine 413 may define the lumen 412, and further define the suction ports 406 in fluid communication with the lumen. The intrauterine portion 402 may be a biased or pre-stressed member configured to at least partially bend and/or curl when ejected from the bore 438. The biasing may be facilitated through material properties from which the intrauterine portion 402 is formed, or with a subcomponent such as an internal stylet formed from shape-memory material. With the slider 418 in the first position, the segments 409 are positioned adjacent to one another and the intrauterine portion 402 is constrained by the bore 438 in a straight configuration. It is noted that the cervical portion 404 may further define the bore 438 to provide ample clearance to accommodate the intrauterine portion 402 in the undeployed configuration. Further, the cervical seal 426 may be deployed sequentially or simultaneously with deployment of the intrauterine portion 402. As the push input is provided to the slider 418 to move it from the first position to the second position, the intrauterine portion 402 is exposed beyond the distal end 411. The intrauterine portion 402 is biased to cause the segments 409 to pivot relative to one another about the spine 413 into a hook shape with the suction ports 406 positioned on an interior of the hook shape. It is contemplated that the hook shape may not be generally circular as shown, but may take another arcuate contours complementary to the shape of the uterus. For example, the biasing member may be formed with a compound bend to provide for more intricate deployment configurations. For another example, the biasing member may be formed to provide for the intrauterine portion 402 to “double-back” on itself to form a helix of any number of revolutions.

It is noted that the slider 418 may be disposed on a same side to which the intrauterine portion 402 is biased. As a result, the user may observe the orientation of the slider 418 and readily appreciated the direction to which the intrauterine portion 402 is to be deployed. Further, the device 400 may include the indicia 422 that may include numerical information as to the angle being achieved by the intrauterine portion 402 in the deployed configuration. For example, the indicia 422 of the illustrated implementation may inform the user the intrauterine portion 402 is deployed by approximately 330 degrees. Together with the orientation of the slider 418, the user may ascertain the shape and position of the intrauterine portion 402 within the uterus.

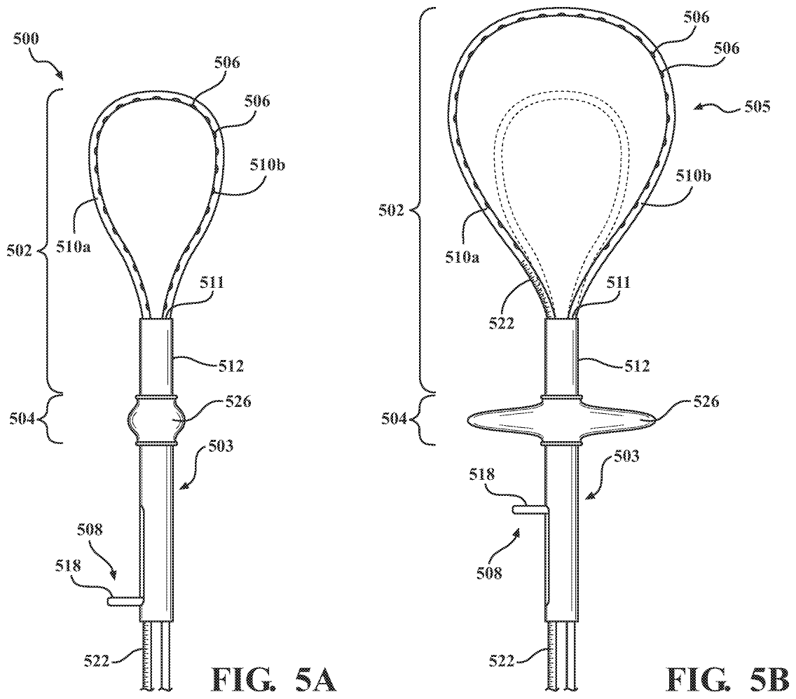

FIGS. 5A and 5B show another implementation of the device 500 in which the intrauterine portion 502 is configured to be actuated via a push input. The intrauterine portion 502 includes an adjustable loop 505. The loop 505 may be formed from resilient biocompatible material with a distal end of the loop 505 being rounded. Other shapes are contemplated so as to be complementary to the shape of the uterus. The loop 505 defines the lumen (not identified), and the suction ports 506 in fluid communication with the lumen. The suction ports 506 are shown as equally spaced on an inner side of the loop 505, but other positions and arrangements are contemplated as mentioned.

The loop 505 may include a first opposing member 510a fixedly coupled to the device body 503, and a second opposing member 510b that is slidably within the device body 503 (and the cervical portion 504). The actuator 508 may be the slider 518 operably coupled to the second opposing member 510b. The slider 518 is configured to be translated relative to the device body 503 to cause the loop 505 to be further exposed beyond the distal end 511. An extent by which the slider 518 is actuated correspondingly provides for the extent by which the outer profile of the intrauterine portion 502 increases in the deployed configuration. FIG. 5B shows the loop 505 generally remaining symmetric about the longitudinal axis of the device 500 in the deployed configuration, but the structure of the loop 505 may be designed to provide for off-axis deployment. For example, the loop 505 may be formed from materials or sections of varying stiffness such that, when exposed beyond the distal end, assume a desired shape. As an alternative to the loop 505 as shown, the loop 505 may be formed from segments pivotably joined by a spine (see FIG. 4A and 4B).

The device 500 may include the indicia 522 disposed on a portion of the loop 505 and positioned to be observable external to the patient as the intrauterine portion 502 is being deployed to the deployed configuration. Based on the indicia 522, the user may ascertain the size of the outer profile of the intrauterine portion 502 within the uterus. The device 500 further includes the cervical portion 504 and the cervical seal 526 to be deployed in the aforementioned manners. It is contemplated that more than one loop may be provided. In such an arrangement, the actuator 508 may be operably coupled to the loops to deploy them simultaneously and to the same extent, or more than one actuator may be operably coupled to a respective one of the loops to selectively deploy the loops to different extents.

In one variant, both the first and second opposing members 510a, 510b are slidably within the device body 503 with the actuator 508 coupled to the first and second opposing members 510a, 510b. Alternatively, more than slider 518 may be coupled to a respective one of the first and second opposing members 510a, 510b. In such a variant, the each of the sliders 518 may be selectively adjusted by the same or differing amounts to selectively tune the resulting shape of the loop 505. For example, the user may push one of the sliders 518 and pull another one of the sliders 518 to provide for an asymmetric deployment of the intrauterine portion 502 within the uterus. Such functionality may be particularly advantages for clinical scenarios in which focal bleeding is identified on one side of the uterus.

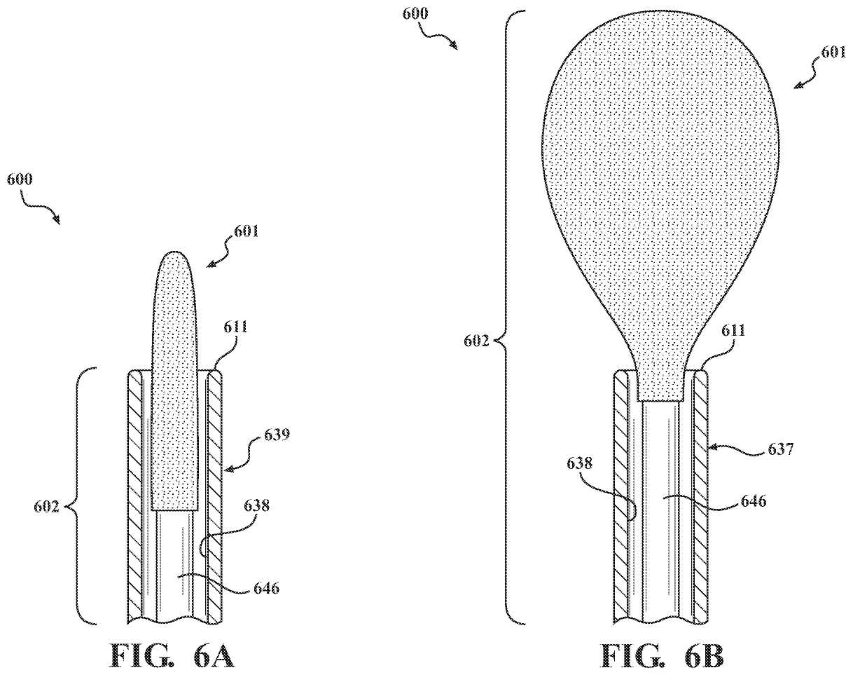

Referring to FIG. 6A and 6B, another implementation of the device 600 is shown in which the intrauterine portion 402 is configured to be actuated via a push input. The device 600 includes an applicator 637 defining a bore 638 sized to receive a head 601 of the intrauterine portion 602. The head 601 may be formed from a porous medium, such as a resiliently compressible biocompatible foam. The head 601 may be coupled to an inner tube 646 defining a lumen in fluid communication with the foam. The head 601 is compressible so as to be fully received within the applicator 637 in the undeployed configuration. The applicator 637 has an outer diameter that is sufficiently narrow to aid with insertion of the device 600 through the body opening. An actuator (not shown), such as the slider previously described, may be operably coupled to the inner tube 646. The actuator is configured to receive the input from the user to move from the first position to the second position to correspondingly move the inner tube 646 and the head 601 distally relative to the applicator 637. As the head 601 is exposed beyond the distal end 611 of the applicator 637, the resilience of the foam causes the outer profile of the head 601 to increase, thereby moving the intrauterine portion 602 to the deployed configuration. FIG. 6B shows the head 601 as assuming a pear-shape complementary to the shape of the uterus, but the head 601 may be designed to assume other suitable shapes. Particularly due to the compressibility and manufacturability of the foam, highly eccentric and irregular shapes may be achieved. For example, a distal section of the head 601 may be two or more times wider than a proximal section in the deployed configuration. For another example, the proximal section may be wider than the distal section in the deployed configuration for clinical instances in which it may be indicated to absorb blood pooling near the cervix. It is also contemplated that stiffening elements and other structures may be coupled to or embedded in the foam so as to cause deployment of a desired shape.

In the deployed configuration, the foam has absorbency to draw the blood into the head 601. Further, the foam may be formed with a density to permit the suction to be drawn through the head 601. Additional suction may be configured to be drawn through the applicator 637, which may be particularly advantageous due to the distal end 611 of the applicator 637 being positioned just distal to the cervix where blood may be more likely to accumulate. It should be appreciated that the present implementation of the device 600 may include the cervical portion, the auxiliary suction source, and other features of the other implementations described herein.

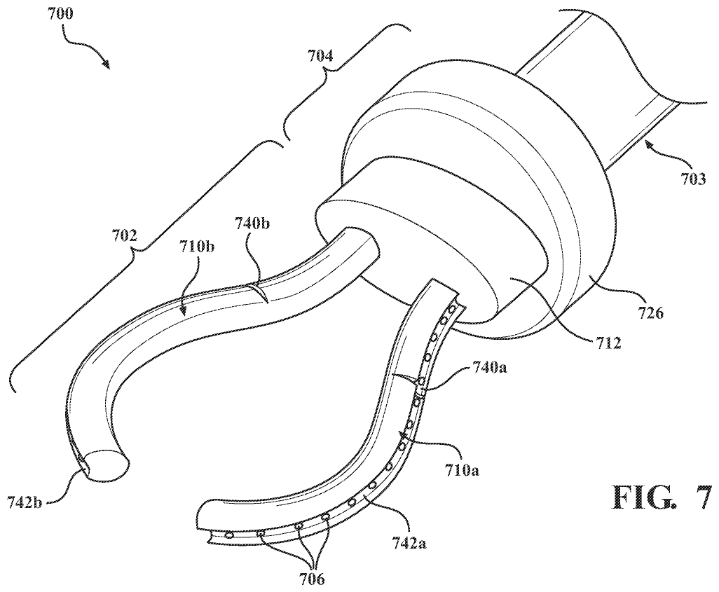

FIG. 7 shows another implementation of the device 700 in which the intrauterine portion 702 is configured to be actuated via a pull input or a push input. The intrauterine portion 702 may include first and second opposing members 710a, 710b that, unlike the loop, each terminate at a respective distal end. The first and second opposing members 710a, 710b may be considered tines. It should be understood that there may be more than two tines arranged in any suitable radial orientations. For example, the tines may be equiangularly spaced radially about the collar 712, or irregularly spaced so as to provide a focal concentration of tines considered to be situated in areas of the uterus in which excessive bleeding is more likely to occur.

The first and second opposing members 710a, 710b each include a proximal end coupled to the collar 712, and a living hinge 740a, 740b between the proximal end and the distal end. The living hinges 740a, 740b are configured to permit a distal section of the opposing members 710 to pivot relative to a proximal section. In other words, the opposing members 710 may be “foldable.” The distal sections may be arcuate and flared outwardly to form an outer profile of the intrauterine portion 702 that is at least partially complementary to the uterus.

In one variant, the opposing members 710 are formed or biased to be fully extended as shown in FIG. 7. The opposing members 710 may be formed from resilient biocompatible material. The distal sections (i.e., distal to the living hinges 740) of the opposing members 710 are operably coupled to the actuator (not identified), for example, through a pull wire. The actuator is actuated to move the device 700 to the undeployed configuration in which the distal sections pivot inwardly about the living hinges 740 to create a narrower outer profile of the intrauterine portion 702. The input may be maintained on the actuator as the intrauterine portion 702 is inserted through the body opening and to within the uterus. Once positioned as desired, the input to the actuator may be removed, and the resiliency of the opposing members 710 causes the distal sections to pivot outwardly about the living hinges 740 for the opposing members 710 to return to their natural state, thereby moving the device 700 to the deployed configuration.

In another variant, the opposing members 710 are biased for the distal sections to be folded inwardly. The device 700 is in the undeployed configuration and insertable through the body opening. The actuator is actuated to cause the distal sections to pivot outwardly about the living hinges 740, thereby moving the device 700 to the deployed configuration. For example, stiffening elements may be slidably positioned within bores extending through a respective one of the opposing members 710. A push input to the actuator causes the stiffening elements to be urged distally within the bores to straighten the opposing members 710. It is contemplated that, in either variant, it is contemplated that the first and second opposing members 710a, 710b may independently deployable. More than one actuator may be provided with each of the actuators being coupled to a respective one of the first and second opposing members 710a, 710b.

The opposing members 710 define the suction ports 706. In the present implementation, the opposing members 710 define channels 742, wherein the suction ports 706 are disposed within the channels. The positioning of the suction ports 706 within the channels 742 is desirable in instances where the suction ports 706 are on outer surfaces of the device 700, such as shown in FIG. 7. In other words, the outer surfaces of the opposing members 710 are likely to contact the tissue of the uterus, and thus tissue suction would otherwise be more likely with the suction ports 706 on the outer surfaces. Therefore, the channels 742 provide a gap or clearance that may limit instances of tissue suction. Additionally or alternatively, the suction ports 706 may be disposed on the inner surfaces of the opposing members 710. The suction ports 706 may be equally spaced, or more or less suction ports may be disposed on the distal sections or the proximal sections. The opposing members 710 further define lumens in fluid communication with the suction ports 706. The lumens may be merged within the device 700 to form a single suction path, or each of the lumens may remain fluidly separate with the device 700 configured to independently control suction through each of the opposing members 710. It should be further appreciated that the present implementation of the device 600 may include the cervical portion, the auxiliary suction source, and other features of the other implementations described herein.

A sequalae of postpartum hemorrhage may include the formation of blood clots within the uterus. The blood clots may be of sufficient size to occlude or clog one or more of the suction ports, thereby possibly compromising the effect of the suction. The actuation-based functionality of the devices of the present disclosure may also be realized for active clog management. One implementation of the device 800 is shown in FIGS. 8A-8D in which the actuator (not identified) is actuated to selectively permit and prevent suction through one or more the suction ports 806. The intrauterine portion 802 includes a head 844 defining the suction ports 806, and an inner tube 846 rotatably disposed within the head 844. The head 844 comprises flanges 845 defining channels with the suction ports 806 disposed within the channels for reasons previously explained. The illustrated implementation shows the intrauterine portion 802 as elongate with a generally consistent cross-section, but the head 844, such as a main body or the flanges 845 may flare outwardly or be of any suitable contour to provide a varied cross-sectional profile to the intrauterine portion 802. Likewise, the suction ports 806 may be positioned on any of the surfaces of the head 844 and in any suitable arrangement.

The inner tube 846 may define a lumen configured to be arranged in fluid communication with the suction path. The inner tube 846 may define one or more openings 848 in fluid communication with the lumen. With the inner tube 846 rotatable within the head 844, the openings 848 are configured to be arranged in selective fluid communication with the suction ports 806. For example, FIG. 8C shows the suction ports 806 being radially blocked on the opposing sides of the head 844, with a single one of the openings 848 in alignment with an upper one (row) of the suction ports 806. Suction within the uterus is established through the alignment, and suction through the other two suction ports 806 may be prevented. A blood clot (C) may become lodged against the suction port 806, and a measured drop in suction, as measured on the medical waste collection system 100, may be interpreted as clogging of the suction ports 806.

As a result, the user may provide the input to the actuator (not identified) to rotate the inner tube 846 within the head 844. As shown in FIG. 8D, the relative rotation moves the opening 848 into alignment with another one of the suction ports 806. The alignment reestablishes the suction through an unclogged one of the suction ports 806, and prevents suction through the clogged one of the suction ports 806. In addition to maintaining the desired level and effect of suction within the uterus, the discontinuation of the suction through the clogged suction port may result in the blood clot becoming dislodged. The previously clogged suction port 806 may again become patent for subsequent use if desired.

It is understood that the inner tube 846 may include more than one opening 848. For example, the inner tube 846 may include two openings on opposing sides configured to align with suction ports 806 on opposing sides of the head 844. For another example, the inner tube 846 may include an equal number of openings and configured to be aligned with all of the suction ports 806 on the head 844. In such an example, rotation of the inner tube 846 within the head 844 is configured to selective activate or deactivate the suction through the head 844 through the inputs to the actuator. It is further contemplated that the active clog management of the present implementation may be realized in any other of the implementations described herein. For example, inner tubes defining the openings may be disposed within the various opposing members (e.g., loop, tines, etc.) described herein, and operably coupled to additional actuators. Inputs to those actuators may rotate the inner tube within the opposing members to selectively prevent suction through one or more of the suction ports that may be clogged.

Another implementation for reducing clogging or its effects is shown in FIG. 9. The intrauterine portion 902 may include a shell 956 that defines a volume, and apertures 958 in fluid communication with the volume. A head 905 includes a face 907 that defines the suction ports 906. The shell 956 is coupled to the head 905 to define the volume with the suction ports 906 opening into the volume. The suction ports 906 may be further defined by funnels 952 extending inwardly from the face, and protrusions 954 extending outwardly from the face 907.

The shell 956 is configured to provide a first barrier to capture blood clots above a first size. As shown in FIG. 9, a blood clot is captured by one of the apertures 958 of the shell 956, whereas the suction through the remaining apertures 958 remain unobstructed. In other words, the suction remains unimpeded through the other apertures 958 radially disposed about the shell 956. It is also noted that the apertures 958 are elongate in dimension and therefore less likely to be occluded by a blood clot that may be generally spherical in shape. Should the blood clot be small enough to pass through one of the apertures 958 and into the volume, the funnels 952 and/or the protrusions 954 are configured to provide a second barrier to limit compromise of the effect of suction. For example, the funnels 952 may cause the blood clot to at least partially collect within the funnel 952, thereby making it less likely the blood clot clogs multiple suction ports. Likewise, the protrusions 954 may guide the blood clot towards the funnels 952. Further, the suction port 906 being defined through the protrusion 954, for example at a distal end of a frustrum of a cone, renders it unlikely that the blood clot would be situated on the distal end of the frustrum. Whereas FIG. 8 may be considered a form of active clog management, FIG. 9 may be a form of passive clog management.

Several configurations have been discussed in the foregoing description. However, the configurations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A device for treating bleeding within a uterus, the device comprising:
a device body comprising a vacuum connector configured to be removably coupled to a suction tube;
an intrauterine portion coupled to the device body and defining a lumen, and suction ports in fluid communication with the lumen, wherein the intrauterine portion has an outer profile in an undeployed configuration that is sized and shaped to be directed through a body opening and positioned within the uterus; and
an actuator coupled to the device body and configured to receive an input to be deployed to a deployed configuration in which the outer profile of the intrauterine portion increases in size within the uterus.

2. The device of claim 1, wherein the intrauterine portion comprises opposing members coupled to the actuator and defining the suction ports, wherein the opposing members are biased to be adjacent to one another in the undeployed configuration, and configured to be moved apart from one another in the deployed configuration based on the input to the actuator.

3. (canceled)

4. The device of claim 2, wherein the intrauterine portion further comprises a collar to which the opposing members are coupled, and a baffle coupled to the collar, wherein the baffle is positioned between the opposing members and configured to occlude the suction ports in the undeployed configuration.

5. The device of claim 1, wherein the intrauterine portion comprises at least two tines each coupled to the actuator and defining the suction ports, and wherein in the tines are configured to be rotated relative to one another for the tines to be splayed outside of a single reference plane in the deployed configuration based on the input to the actuator.

6. The device of claim 1, wherein the intrauterine portion comprises a pre-stressed member coupled to the actuator and defining the suction ports, and wherein the pre-stressed member is configured to be ejected from the device body to assume a hook shape in the deployed configuration based on the input to the actuator.

7. The device of claim 6, wherein the pre-stressed member comprises segments coupled to one another with a spine, wherein the spine defines the suction ports.

8. The device of claim 1, wherein the intrauterine portion comprises an adjustable loop comprising a first end fixedly coupled to the device body, and a second end coupled to the actuator for the adjustable loop to be extended from the device body in the deployed configuration based on the input to the actuator.

9. The device of claim 1, wherein the intrauterine portion comprises an applicator, a head formed from compressible foam, and an inner tube coupled to the head and the actuator, and wherein the head is configured to resiliently expand when extended from the applicator based on the input to the actuator.

10. The device of claim 1, wherein the intrauterine portion comprises opposing members coupled to the actuator and defining the suction ports, and wherein each of the opposing members comprises a living hinge about which respective distal sections of the opposing members are configured to pivot relative to proximal sections based on the input to the actuator.

11. The device of claim 1, wherein the actuator comprises a slider or a handle movably coupled to the device body, or a wheel rotatably coupled to the device body.

12. The device of claim 1, wherein the intrauterine portion further defines a channel with the suction ports disposed within the channel; and, optionally, wherein the channel is defined on an outer side of the intrauterine portion.

13-15. (canceled)

16. The device of claim 1, wherein the actuator is configured to be moved between a first position with the intrauterine portion in the undeployed configuration, a second position with the intrauterine portion in the deployed configuration, and positioned between the first and second positions to selectively establish an extent of deployment of the intrauterine portion, and wherein the actuator comprises indicia configured to be exposed to provide information as to the extent of deployment of the intrauterine portion.

17. (canceled)

18. The device of claim 1, wherein the intrauterine portion further comprises an inner tube disposed within the lumen and defining openings, wherein the inner tube is configured to be rotated within the lumen to selectively align the openings with less than all of the suction ports to provide active clog management.

19. A device for treating bleeding within a uterus, the device comprising:
a device body comprising a vacuum connector configured to be removably coupled to a suction tube;
an intrauterine portion defining a lumen configured to be arranged in fluid communication with a vacuum source, and suction ports in fluid communication with the lumen, wherein the intrauterine portion is configured to form a loop in a deployed configuration and comprises a baffle configured to engage and occlude the suction ports in an undeployed configuration; and
an actuator coupled to the device body and configured to receive an input from a user to move the intrauterine portion from the undeployed configuration to the deployed configuration in which an outer profile of the intrauterine portion is increased.

20. The device of claim 19, further comprising a collar to which the loop is coupled, wherein the baffle is coupled to the collar and a distal end of the loop.

21. The device of claim 20, wherein the actuator is coupled to the baffle with the baffle configured to translate within the device body to draw the distal end of the loop proximally.

22. A device for treating bleeding within a uterus, the device comprising:
a device body comprising a vacuum connector configured to be removably coupled to a suction tube;
an intrauterine portion defining a lumen configured to be arranged in fluid communication with a vacuum source, and suction ports in fluid communication with the lumen;
a cervical portion configured to seal a cervix;
an actuator coupled to the device body and configured to receive an input from a user to move the intrauterine portion from an undeployed configuration to a deployed configuration in which an outer profile of the intrauterine portion is increased; and
an auxiliary suction source disposed proximal to the cervical portion and configured to receive a manual input from a user to establish or maintain a vacuum in an absence of a vacuum connector being coupled to a vacuum source.

23. The device of claim 22, further comprising:
a first valve positioned distal to the auxiliary suction source; and
a second valve is positioned proximal to the auxiliary suction source,
wherein the vacuum connector is coupled proximal to the second valve.

24. The device of claim 22, wherein the auxiliary suction source is a bulb configured to receive an input in which resilience of the bulb provides a vacuum, and defines a reservoir to collect bodily fluids.

25. The device of claim 24, further comprising a relief port coupled to the device body and positioned distal to the first valve.

26-30. (canceled)
